(19) United States
(12) Patent Application Publication
(10) Pub. No.: US 2024/0366668 A1
(43) Pub. Date: Nov. 7, 2024

(54) NKG2D CAR CELLS EXPRESSING IL-18 FOR ADOPTIVE CELL THERAPY

(71) Applicant: CELYAD ONCOLOGY S.A., Mont-Saint-Guibert (BE)

(72) Inventor: Eytan BREMAN, Mont-Saint-Guibert (BE)

(21) Appl. No.: 18/580,692

(22) PCT Filed: Jul. 18, 2022
(86) PCT No.: PCT/EP2022/070090

§ 371 Date: Jan. 19, 2024

(30) Foreign Application Priority Data
Jul. 19, 2021 (GB) 2110363.5

(51) Int. Cl. A61K 35/17; A61K 39/00; A61P 35/00; C07K 14/54; C07K 14/705; C12N 15/85

(52) U.S. Cl. CPC A61K 35/17; A61K 39/4611; A61K 39/4613; A61K 39/464402; A61K 39/46444; A61P 35/00; C07K 14/54; C07K 14/7056; C12N 15/85

(57) ABSTRACT

The present application relates to the field of immunotherapy, more particularly to the field of adoptive cell therapy (ACT). Here, NKG2D CAR expressing cells that also express IL-18 are proposed. Also proposed are polynucleotides, vectors encoding the NKG2D CAR and IL-18 and cells expressing both. These cells are particularly suitable for use in immunotherapy, including allogeneic therapy. The invention provides methods of increasing the efficacy of a T cell therapy in a patient in need thereof. Further, strategies to treat diseases such as cancer using these cells are also provided. The engineered immune cells, such as T-cells or natural killer (NK) cells, expressing such CARs are suitable for treating lymphomas, multiple myeloma and leukemia, but other tumors can be treated as well, depending on the specificity of the CAR. Most notably, as expression and secretion of the pro-inflammatory cytokine IL-18 is beneficial to reducing the immunosuppressive effect of the tumor microenvironment, treatment of solid tumors is envisaged.

### FIELD OF THE INVENTION

The present application relates to the field of immunotherapy, more particularly to the field of adoptive cell therapy (ACT). Here, NKG2D CAR expressing cells that also express IL-18 are proposed. Also proposed are polynucleotides, vectors encoding the NKG2D CAR and IL-18 and cells expressing both. These cells are particularly suitable for use in immunotherapy, including allogeneic therapy. The invention provides methods of increasing the efficacy of a T cell therapy in a patient in need thereof. Further, strategies to treat diseases such as cancer using these cells are also provided. The engineered immune cells, such as T-cells or natural killer (NK) cells, expressing such CARs are suitable for treating lymphomas, multiple myeloma and leukemia, but other tumors can be treated as well, depending on the specificity of the CAR. Most notably, as expression and secretion of the pro-inflammatory cytokine IL-18 is beneficial to reducing the immunosuppressive effect of the tumor microenvironment, treatment of solid tumors is envisaged.

### BACKGROUND

The recent FDA approval of the first two CAR-T therapies, both directed against the B cell antigen CD19, has led to an ever-increasing interest in the CAR-T field. CARs are artificial antigen binding receptors, engineered to recognize cancer antigens through their antigen binding domain, resulting in the activation of the CAR cell through the intracellular signaling domain of the receptor. CD19 directed CARs are useful for treatment of liquid tumors (due to the specific expression pattern of CD19). Treatment of solid tumors is highly desirable, but hampered by the fact that there are very few antigens expressed on tumors that are not present in healthy tissue. One approach that is of potential interest is targeting stress-induced ligands, ligands that are not present on healthy tissue, but are a hallmark of stressed or cancerous cells. An example of a CAR T that falls in this category is a CAR based on a chimeric NKG2D receptor (U.S. Pat. No. 7,994,298). Such CAR has promise in treatment of both liquid and solid tumors.

A common problem for cell-based cancer therapies is that cancer cells adapt to generate an immunosuppressive microenvironment to protect themselves from immune recognition and elimination. Overcoming this immunosuppressive tumor microenvironment poses a significant hurdle. Several options have been proposed to address this challenge, one of which is to let the CAR T cell secrete factors that protect or “armor” the T cells from the suppressive tumor microenvironment, such as cytokines. Several cytokines have been proposed to this end, including e.g. IL-2 (Heemskerk et al., 2008), IL-12 (Chmielewski et al., 2011, Pegram et al., 2012, Zhang et al., 2015), IL-15 (Hurton et al., 2016; US20130071414), interferon-β (IFN-β) (Zhao et al., 2015), IL-18 (Chmielewski and Abken, 2017), IL-7 (Golumba-Nagy et al., 2018), IL-7 and CCL19 (Adachi et al., 2018), IL-36γ (Li et al., 2021). However, many of these approaches are associated with significant toxicity.

Accordingly, there is a need for therapies that can alter the tumor microenvironment, can target a variety of tumors, and are more efficient and safe than the present solutions.

### SUMMARY

In the current application, we compared and assessed the effect of armoring a NKG2D CAR with the cytokine IL-18. It was surprisingly shown that this resulted in a significant reduction of tumor growth compared to treatment with the NKG2D CAR as such, coupled with a significant increase in survival. Moreover, this effect was observed independent of other additions, such as shRNA against stress ligands, and was observed both in autologous and allogeneic CARs (wherein the TCR has been downregulated). The effect appears not strictly dependent on tumor microenvironment, as it is observed both in solid and hematological tumors. The increased survival indicates that this approach both is more efficient and lacks significant toxicity.

Accordingly, provided herein are engineered cells comprising:
A first exogenous nucleic acid molecule encoding a chimeric NKG2D receptor
a second nucleic acid molecule encoding IL-18.

Typically, the cells are engineered immune cells. According to particular embodiments, the immune cell is selected from a T cell, a NK cell, a NKT cell, a stem cell, a progenitor cell, and an iPSC cell.

According to particular embodiments, the first and second nucleic acid molecule are present in one vector, such as a eukaryotic expression plasmid, a mini-circle DNA, or a viral vector (e.g. derived from a lentivirus, a retrovirus, an adenovirus, an adeno-associated virus, and a Sendai virus).

According to particular embodiments, the promoter used for IL-18 expression is the same as that for expression of the NKG2D CAR. According to alternative embodiments, IL-18 is expressed using a separate promoter. In both instances, the promoter can be a constitutive promoter or an inducible promoter. It is particularly envisaged that the encoded IL-18, once expressed, is secreted. To this end, the nucleic acid molecule encoding IL-18 may further contain a secretion signal.

According to particular embodiments, the engineered cell has been further engineered to reduce or inactivate TCR signalling. According to specific embodiments, TCR signalling has been inactivated through gene editing. According to alternative specific embodiments, TCR signalling has been reduced through RNA interference. According to further specific embodiments, the RNA interference to reduce TCR signalling comprises a RNA interference molecule directed against a TCR receptor complex subunit (such as e.g. CD247 or the TCR alpha chain).

According to a further aspect, the engineered cell as described herein is provided for use as a medicament. Particularly, the cell as described herein is provided for use in the treatment of cancer.

This is equivalent as stating that methods of treating cancer are provided, comprising administering to a subject in need thereof a suitable dose of cells as described herein, thereby improving at least one symptom.

According to alternative embodiments, the cells as described herein are provided for use in the treatment of infectious disease, e.g. viral infection. According to alternative embodiments, the cells are provided for use in the treatment of autoimmune disease.

This is equivalent as stating that methods of treating infectious disease are provided, comprising administering to a subject in need thereof a suitable dose of cells as described herein, thereby improving at least one symptom. Or, alternatively, that methods of treating autoimmune disease are provided, comprising administering to a subject in need thereof a suitable dose of cells as described herein, thereby improving at least one symptom.

The engineered cells may be autologous immune cells (cells obtained from the patient) or allogeneic immune cells (cells obtained from another subject), with the latter being particularly envisaged.

### BRIEF DESCRIPTION OF THE FIGURES

FIG. 1. Schematic representation of the constructs used in Example 1 and 2. From top to bottom: NKG2D CAR, NKG2D CAR shMICA/B (with an additional shRNA against MICA and MICB ligands), NKG2D armored CAR (with IL-18), NKG2D armored CAR shMICA/B (with both IL-18 and an additional shRNA against MICA and MICB ligands).

FIG. 2. Culture data of T cells transduced with the CAR constructs of FIG. 1. A. CD4/CD8 ratio; B. IL-18 secretion; C. Fold expansion.

FIG. 3. CAR efficacy upon coculture of K562 cells. A. IFN-γ secretion of CAR-T, CAR-T in presence of K562 cells and CAR-T in presence of K562 cells and a NKG2D blocking antibody. B. IL-18 secretion of CAR-T in presence of K562 cells and CAR-T in presence of K562 cells and a NKG2D blocking antibody.

FIG. 4. repeated antigen stimulation assay: relative PANC-1 counts for the different constructs indicated over time. A. First stimulation. B. Second stimulation, using same CAR-T and new PANC-1 cells after 48 hours. C. Third stimulation, using same CAR-T and new PANC-1 cells 48 hours after B. D. Cytokine secretion profile of the different cells after the third stimulation.

FIG. 5. Functionality of secreted IL-18. A) HEK-IL18 cells were incubated with IL-18, sera from samples and/or IL-18BP which inhibits interaction between IL-18 and its receptor. Addition of IL-18 through either the supernatans (SN) or in recombinant form induced HEK SEAP activity, which was inhibited in the presence of IL18BP. B) IL-18 secreted by armoured NKG2D CAR T cells was functionally assessed with the HEK-IL18 cell line. All armored CAR T cells induced a specific enzymatic response that was inhibited by IL18BP thereby indicating the secreted IL-18 is indeed functional and can interact with its receptor.

FIG. 6. Bioluminescence data of NSG mice orthotopically injected with HTC116 cells followed by repeated injection of NKG2D CAR (top panels) or NKG2D CAR also expressing IL-18 (bottom panels).

FIG. 7. Tumor growth data and survival data of an in vivo AML experiment. A. Autologous setting: NKG2D CAR compared to NKG2D CAR also expressing IL-18. B. Persistence-enhanced setting: NKG2D CAR co-expressing shMICA/B compared to NKG2D armored CAR shMICA/B. C. Allogeneic setting: NKG2D CAR co-expressing shRNA against CD247 compared to NKG2D armored CAR co-expressing shRNA against CD247.

FIG. 8. NSG mice were injected with THP-1 cancer cells and seven days later infused with vehicle, mock or allogeneic armoured NKG2D CAR T cells. Mice infused with a high dose of allogeneic armoured NKG2D CAR T cells exhibited signs of cytokine release syndromee driven by the secreted IL-18. This effect could be negated by the infusion of IL18BP.

### DETAILED DESCRIPTION

### Definitions

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The following terms or definitions are provided solely to aid in the understanding of the invention. Unless specifically defined herein, all terms used herein have the same meaning as they would to one skilled in the art of the present invention. Practitioners are particularly directed to Green and Sambrook, Molecular Cloning: A Laboratory Manual, 4th ed., Cold Spring Harbor Laboratory Press, New York (2012); and Ausubel et al., Current Protocols in Molecular Biology (up to Supplement 114), John Wiley & Sons, New York (2016), for definitions and terms of the art. The definitions provided herein should not be construed to have a scope less than understood by a person of ordinary skill in the art.

An “engineered cell” as used herein is a cell that has been modified through human intervention (as opposed to naturally occurring mutations).

The phrase “nucleic acid molecule” synonymously referred to as “nucleotides” or “nucleic acids” or “polynucleotide” refers to any polyribonucleotide or polydeoxyribonucleotide, which may be unmodified RNA or DNA or modified RNA or DNA. Nucleic acid molecules include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

A “chimeric antigen receptor” or “CAR” as used herein refers to a chimeric receptor (i.e. composed of parts from different sources) that has at least a binding moiety with a specificity for an antigen (which can e.g. be derived from an antibody, a receptor or its cognate ligand) and a signaling moiety that can transmit a signal in an immune cell (e.g. a CD3 zeta chain. Other signaling or cosignaling moieties can also be used, such as e.g. a Fc epsilon RI gamma domain, a CD3 epsilon domain, the recently described DAP10/DAP12 signaling domain, or domains from CD28, 4-1BB, OX40, ICOS, DAP10, DAP12, CD27, and CD2 as costimulatory domain). A “chimeric NK receptor” is a CAR wherein the binding moiety is derived or isolated from a NK receptor. A “chimeric NKG2D receptor” is a CAR wherein the binding moiety is derived or isolated from a NKG2D receptor, such as described in e.g. U.S. Pat. No. 7,994,298 or US2016/0000828.

A “TCR” as used herein refers to a T cell receptor. In the context of adoptive cell transfer, this typically refers to an engineered TCR, i.e. a TCR that has been engineered to recognize a specific antigen, most typically a tumor antigen. An “endogenous TCR” as used herein refers to a TCR that is present endogenously, on non-modified cells (typically T cells). The TCR is a disulfide-linked membrane-anchored heterodimeric protein normally consisting of the highly variable alpha (α) and beta (β) chains expressed as part of a complex with the invariant CD3 chain molecules. The TCR receptor complex is an octomeric complex of variable TCR receptor α and β chains with the CD3 co-receptor (containing a CD3γ chain, a CD3δ chain, and two CD3ε chains) and two CD3ζ chains (aka CD247 molecules). The term “functional TCR” as used herein means a TCR capable of transducing a signal upon binding of its cognate ligand. Typically, for allogeneic therapies, engineering will take place to reduce or impair the TCR function, e.g. by knocking out or knocking down at least one of the TCR chains. An endogenous TCR in an engineered cell is considered functional when it retains at least 50%, at least 60%, at least 70%, at least 75%, at least 80%, or even at least 90% of signalling capacity (or T cell activation) compared to a cell with endogenous TCR without any engineering. Assays for assessing signalling capacity or T cell activation are known to the person skilled in the art, and include amongst others an ELISA measuring interferon gamma. According to alternative embodiments, an endogenous TCR is considered functional if no engineering has taken place to interfere with TCR function.

A “transmembrane domain” or “TM domain” as used herein is any membrane-spanning protein domain. Most typically, it is derived from a transmembrane protein. However, it can also be artificially designed. Transmembrane domains used herein will typically associate with other transmembrane domains, through charged and non-charged interactions.

The term “signaling domain” as used herein refers to a moiety that can transmit a signal in a cell, particularly in an immune cell. The signaling domain typically comprises a domain derived from a receptor that signals by itself in immune cells, such as the T Cell Receptor (TCR) complex or the Fc receptor. Additionally, it may contain a costimulatory domain (i.e. a domain derived from a receptor that is required in addition to the TCR to obtain full activation, or the full spectrum of the signal in case of inhibitory costimulatory domains, of T cells). The costimulatory domain can be from an activating costimulatory receptor or from an inhibitory costimulatory receptor.

The term “immune cells” as used herein refers to cells that are part of the immune system (which can be either the adaptive or the innate immune system). Immune cells as used herein are typically immune cells that are manufactured for adoptive cell transfer (either autologous transfer or allogeneic transfer). Many different types of immune cells are used for adoptive therapy and thus are envisaged for use in the methods described herein. Examples of immune cells include, but are not limited to, T cells, NK cells, NKT cells, lymphocytes, dendritic cells, myeloid cells, stem cells, progenitor cells or iPSCs. The latter three are not immune cells as such, but can be used in adoptive cell transfer for immunotherapy (see e.g. Jiang et al., Cell Mol Immunol 2014; Themeli et al., Cell Stem Cell 2015). Typically, while the manufacturing starts with stem cells or iPSCs (or may even start with a dedifferentiation step from immune cells towards iPSCs), manufacturing will entail a step of differentiation to immune cells prior to administration. Stem cells, progenitor cells and iPSCs used in manufacturing of immune cells for adoptive transfer (i.e., stem cells, progenitor cells and iPSCs or their differentiated progeny that are transduced with a CAR as described herein) are considered as immune cells herein. According to particular embodiments, the stem cells envisaged in the methods do not involve a step of destruction of a human embryo.

Particularly envisaged immune cells include white blood cells (leukocytes), including lymphocytes, monocytes, macrophages and dendritic cells. Particularly envisaged lymphocytes include T cells, NK cells and B cells, most particularly envisaged are T cells. In the context of adoptive transfer, note that immune cells will typically be primary cells (i.e. cells isolated directly from human or animal tissue, and not or only briefly cultured), and not cell lines (i.e. cells that have been continually passaged over a long period of time and have acquired homogenous genotypic and phenotypic characteristics). According to specific embodiments, immune cells will be primary cells (i.e. cells isolated directly from human or animal tissue, and not or only briefly cultured) and not cell lines (i.e. cells that have been continually passaged over a long period of time and have acquired homogenous genotypic and phenotypic characteristics). According to alternative specific embodiments, the immune cell is not a cell from a cell line.

“Isolated” as used herein means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids. An “isolated” antibody or antigen-binding fragment, as used herein, is intended to refer to an antibody or antigen-binding fragment which is substantially free of other antibodies or antigen-binding fragments having different antigenic specificities (for instance, an isolated antibody that specifically binds to BCMA is substantially free of antibodies that specifically bind antigens other than BCMA). An isolated antibody that specifically binds to an epitope, isoform or variant of BCMA may, however, have cross-reactivity to other related antigens, for instance from other species (such as BCMA species homologs).

A “vector” is a replicon, such as plasmid, phage, cosmid, or virus in which another nucleic acid segment may be operably inserted so as to bring about the replication or expression of the segment. A “clone” is a population of cells derived from a single cell or common ancestor by mitosis. A “cell line” is a clone of a primary cell that is capable of stable growth in vitro for many generations. In some examples provided herein, cells are transformed by transfecting the cells with DNA.

The terms “express” and “produce” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications.

The term “exogenous” as used herein, particularly in the context of cells or immune cells, refers to any material that is present and active in an individual living cell but that originated outside that cell (as opposed to an endogenous factor). The phrase “exogenous nucleic acid molecule” thus refers to a nucleic acid molecule that has been introduced in the (immune) cell, typically through transduction or transfection. The term “endogenous” as used herein refers to any factor or material that is present and active in an individual living cell and that originated from inside that cell (and that are thus typically also manufactured in a non-transduced or non-transfected cell).

A “promoter” as used herein is a regulatory region of nucleic acid usually located adjacent to a gene region, providing a control point for regulated gene transcription.

The term “subject” refers to human and non-human animals, including all vertebrates, e.g., mammals and non-mammals, such as non-human primates, mice, rabbits, sheep, dogs, cats, horses, cows, chickens, amphibians, and reptiles. In most particular embodiments of the described methods, the subject is a human.

The terms “treating” or “treatment” refer to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations.

The phrase “adoptive cellular therapy”, “adoptive cell transfer”, or “ACT” as used herein refers to the transfer of cells, most typically immune cells, into a subject (e.g. a patient). These cells may have originated from the subject (in case of autologous therapy) or from another individual (in case of allogeneic therapy). The goal of the therapy is to improve immune functionality and characteristics, and in cancer immunotherapy, to raise an immune response against the cancer. Although T cells are most often used for ACT, it is also applied using other immune cell types such as NK cells, lymphocytes (e.g. tumor-infiltrating lymphocytes (TILs)), dendritic cells and myeloid cells.

An “effective amount” or “therapeutically effective amount” refers to an amount effective, at dosages and for periods of time necessary, to achieve a desired therapeutic result. A therapeutically effective amount of a therapeutic, such as the transformed immune cells described herein, may vary according to factors such as the disease state, age, sex, and weight of the individual, and the ability of the therapeutic (such as the cells) to elicit a desired response in the individual. A therapeutically effective amount is also one in which any toxic or detrimental effects of the therapeutic are outweighed by the therapeutically beneficial effects.

The phrase “graft versus host disease” or “GvHD” refers to a condition that might occur after an allogeneic transplant. In GvHD, the donated bone marrow, peripheral blood (stem) cells or other immune cells view the recipient's body as foreign, and the donated cells attack the body. As donor immunocompetent immune cells, such as T cells, are the main driver for GvHD, one strategy to prevent GvHD is by reducing (TCR-based) signaling in these immunocompetent cells, e.g. by directly or indirectly inhibiting the function of the TCR complex.

A “RNA interference molecule” as used herein is a molecule that mediates RNA interference (RNAi). Several mechanisms of RNAi gene modulation exist in plants and animals. A first is through the expression of small non-coding RNAs, called microRNAs (“miRNAs”). miRNAs are able to target specific messenger RNAs (“mRNA”) for degradation, and thereby promote gene silencing. Small interfering RNAs (“siRNAs”), which are artificially designed molecules, can also mediate RNAi. siRNAs can cause cleavage of a target molecule, such as mRNA, and similar to miRNAs, in order to recognize the target molecule, siRNAs rely on the complementarity of bases.

Within the class of molecules that are known as siRNAs are short hairpin RNAs (“shRNAs”). shRNAs are single stranded molecules that contain a sense region and an antisense region that is capable of hybridizing with the sense region. shRNAs are capable of forming a stem and loop structure in which the sense region and the antisense region form part or all of the stem. One advantage of using shRNAs is that they can be delivered or transcribed as a single molecule, which is not possible when an siRNA has two separate strands. However, like other siRNAs, shRNAs still target mRNA based on the complementarity of bases. A difference between shRNA molecules and miRNA molecules is that miRNA molecules are processed by Drosha, while conventional shRNA molecules are not (which has been associated with toxicity, Grimm et al., Nature 441:537-541 (2006)).

According to a first aspect, provided herein are engineered cells comprising:
A first exogenous nucleic acid molecule encoding a chimeric NKG2D receptor
a second nucleic acid molecule encoding IL-18.

The engineered cells are particularly eukaryotic cells, more particularly engineered mammalian cells, more particularly engineered human cells. According to particular embodiments, the cells are engineered immune cells. Typical immune cells are selected from T cells, NK cells, NKT cells, stem cells, progenitor cells, and iPSC cells.

According to particular embodiments, the first and second nucleic acid molecule are present in one vector, such as a eukaryotic expression plasmid, a mini-circle DNA, or a viral vector (e.g. derived from a lentivirus, a retrovirus, an adenovirus, an adeno-associated virus, and a Sendai virus). Accordingly, also provided herein are vectors that contain a first exogenous nucleic acid molecule encoding a chimeric NKG2D receptor and a second nucleic acid molecule encoding IL-18.

According to particular embodiments, the promoter used for IL-18 expression is the same as that for expression of the NKG2D CAR. According to alternative embodiments, IL-18 is expressed using a separate promoter. In both instances, the promoter can be a constitutive promoter or an inducible promoter. It is particularly envisaged that the encoded IL-18, once expressed, is secreted. To this end, the nucleic acid molecule encoding IL-18 may further contain a secretion signal.

Apart from the NKG2D CAR and the IL-18, the vectors or cells provided herein may comprise further elements that can be useful to improve function. These can be further secreted elements such as cytokines, to further ‘armor’ the CAR. According to particular embodiments, the vectors or cells provided herein further contain a protein (or a nucleic acid encoding a protein). Such protein can be a receptor, or can for instance be a cytokine, chemokine, hormone, antibody, histocompatibility antigen (e.g. HLA-E), a tag, or any other protein of therapeutic or diagnostic value, or for allowing detection. Alternatively or additionally, these can be molecules that block the function of deleterious proteins, or prevent unwanted effects. A prime example of this is the use of RNA interference molecules. Any suitable molecule present in the engineered cell can be targeted by the instant RNA interference molecules. Typical examples of envisaged targets are: a MHC class I gene, a MHC class II gene, a MHC coreceptor gene (e.g. HLA-F, HLA-G), a TCR chain, a CD3 chain, NKBBiL, LTA, TNF, LTB, LST1, NCR3, AIF1, LY6, a heat shock protein (e.g. HSPA1L, HSPA1A, HSPA1B), complement cascade, regulatory receptors (e.g. NOTCH4), TAP, HLA-DM, HLA-DO, RING1, CD52, CD247, HCP5, DGKA, DGKZ, B2M, MICA, MICB, ULBP1, ULBP2, ULBP3, ULBP4, ULBP5, ULBP6, 2B4, A2AR, BAX, BLIMP1, C160 (POLR3A), CBL-B, CCR6, CD7, CD95, CD123, DGK [DGKA, DGKB, DGKD, DGKE, DKGG, DGKH, DGKI, DGKK, DGKQ, DGKZ], DNMT3A, DR4, DR5, EGR2, FABP4, FABP5, FASN, GMCSF, HPK1, IL-10R [IL10RA, IL10RB], IL2, LFA1, NEAT 1, NFkB (including RELA, RELB, NFkB2, NFkB1, REL), NKG2A, NR4A (including NR4A1, NR4A2, NR4A3), PD1, PI3KCD, PPP2RD2, SHIP1, SOAT1, SOCS1, T-BET, TET2, TGFBR1, TGFBR2, TGFBR3, TIGIT, TIM3, TOX, and ZFP36L2. In the context of NKG2D, particularly useful targets for shRNA include one or more of MICA, MICB and a TCR or CD3 chain. Particularly suitable constructs for shRNA expression have been identified which are miRNA-based, such as the miR-196a2 scaffold (Horizon Discoveries), the miR17-92 cluster or the miR-106a˜363 cluster. The latter is particularly suitable when the use of more than one shRNA is envisaged (i.e. multiplexing).

According to particular embodiments, the engineered cell has been further engineered to reduce or inactivate TCR signalling. According to specific embodiments, TCR signalling has been inactivated through gene editing, e.g. using CRISPR or TALEN. According to alternative specific embodiments, TCR signalling has been reduced through RNA interference. According to further specific embodiments, the RNA interference to reduce TCR signalling comprises a RNA interference molecule directed against a TCR receptor complex subunit (such as e.g. CD247 or the TCR alpha chain).

According to a further aspect, the engineered cell as described herein is provided for use as a medicament. Particularly, the cell as described herein is provided for use in the treatment of cancer. Exemplary types of cancer that can be treated include, but not limited to, adenocarcinoma, adrenocortical carcinoma, anal cancer, astrocytoma, bladder cancer, bone cancer, brain cancer, breast cancer, cervical cancer, colorectal cancer, endometrial cancer, esophageal cancer, Ewing sarcoma, eye cancer, Fallopian tube cancer, gastric cancer, glioblastoma, head and neck cancer, Kaposi sarcoma, kidney cancer, leukemia, liver cancer, lung cancer, lymphoma, melanoma, mesothelioma, myelodysplastic syndrome, multiple myeloma, neuroblastoma, osteosarcoma, ovarian cancer, pancreatic cancer, parathyroid cancer, penile cancer, peritoneal cancer, pharyngeal cancer, prostate cancer, renal cell carcinoma, retinoblastoma, rhabdomyosarcoma, sarcoma, skin cancer, small intestine cancer, stomach cancer, testicular cancer, thyroid cancer, urethral cancer, uterine cancer, vaginal cancer, and Wilms tumor.

According to particular embodiments, the cells can be provided for treatment of liquid or blood cancers. Examples of such cancers include e.g. leukemia (including a.o. acute myelogenous leukemia (AML), acute lymphocytic leukemia (ALL), chronic myelogenous leukemia (CML), and chronic lymphocytic leukemia (CLL)), lymphoma (including a.o. Hodgkin's lymphoma and non-Hodgkin's lymphoma such as B-cell lymphoma (e.g. DLBCL), T cell lymphoma, Burkitt's lymphoma, follicular lymphoma, mantle cell lymphoma, and small lymphocytic lymphoma), multiple myeloma or myelodysplastic syndrome (MDS).

This is equivalent as stating that methods of treating cancer are provided, comprising administering to a subject in need thereof a suitable dose of cells as described herein, thereby improving at least one symptom.

According to alternative embodiments, the cells as described herein are provided for use in the treatment of infectious disease, e.g. viral infection. According to alternative embodiments, the cells are provided for use in the treatment of autoimmune disease. Exemplary types of autoimmune diseases that can be treated include, but are not limited to, rheumatoid arthritis (RA), systemic lupus erythematosus (SLE), inflammatory bowel disease (IBD), multiple sclerosis (MS), Type 1 diabetes mellitus, amyotrophic lateral sclerosis (ALS or Lou Gehrig's disease), spinal muscular atrophy (SMA), Crohn's disease, Guillain-Barre syndrome, chronic inflammatory demyelinating polyneuropathy, psoriasis, psoriatic arthritis, Addison's disease, ankylosing spondylitis, Behcet's disease, coeliac disease, Coxsackie myocarditis, endometriosis, fibromyalgia, Graves' disease, Hashimoto's thyroiditis, Kawasaki disease, Meniere's disease, myasthenia gravis, sarcoidosis, scleroderma, Sjögren's syndrome, thrombocytopenia purpura (TTP), ulcerative colitis, vasculitis and vitiligo.

This is equivalent as stating that methods of treating infectious disease are provided, comprising administering to a subject in need thereof a suitable dose of cells as described herein, thereby improving at least one symptom. Or, alternatively, that methods of treating autoimmune disease are provided, comprising administering to a subject in need thereof a suitable dose of cells as described herein, thereby improving at least one symptom.

These cells that are provided for use as a medicament can be provided for use in allogeneic therapies. I.e., they are provided for use in treatments where allogeneic ACT is considered a therapeutic option (wherein cells from another subject are provided to a subject in need thereof). According to specific embodiments, in allogeneic therapies, in addition to the protein of interest and the RNA interference molecule against CD52, the cells will further be engineered to have reduced functional TCR expression (e.g. by genetic knockout, or by expression of an additional molecule, such as a RNA interference molecule, directed against the TCR (most particularly, against a subunit of the TCR complex)). According to alternative embodiments, these cells are provided for use in autologous therapies, particularly autologous ACT therapies (i.e., with cells obtained from the patient).

It is to be understood that although particular embodiments, specific configurations as well as materials and/or molecules, have been discussed herein for cells and methods according to present invention, various changes or modifications in form and detail may be made without departing from the scope and spirit of this invention. The following examples are provided to better illustrate particular embodiments, and they should not be considered limiting the application. The application is limited only by the claims.

### EXAMPLES

### Example 1. Cloning and Characterization of IL-18 Expressing NKG2D CARs

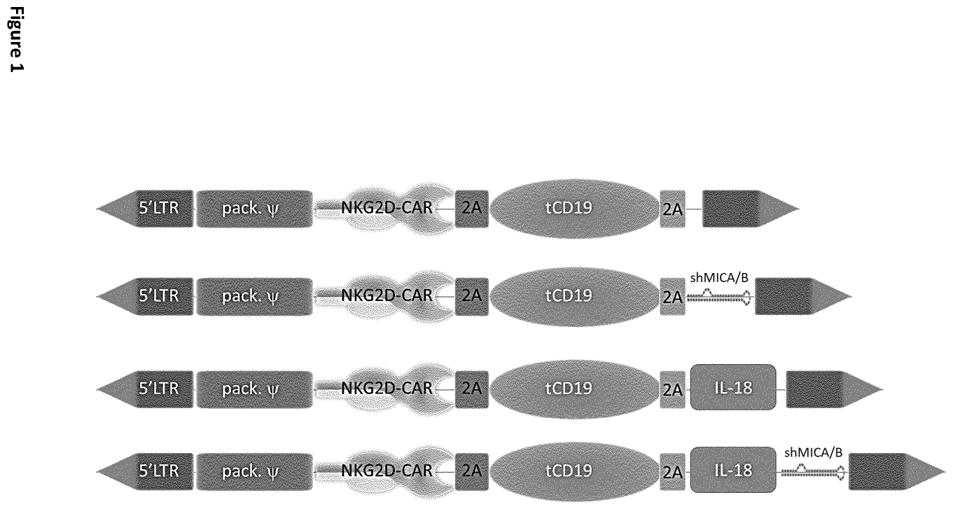

The NKG2D CAR has been described before (U.S. Pat. No. 7,994,298) and has been tested in the clinic. To test whether clinical activity could be further improved, the effect of several additions has been tested. One is the addition of a short hairpin RNA against the NK ligands MICA/MICB. This has been shown before to reduce fratricide of NKG2D-expressing cells (WO2019110667) and to increase persistence of these cells (WO2019110693). Another addition, is the IL-18, creating a so-called ‘armored’ CAR. Both have also been tested in combination. The different constructs are shown in FIG. 1, designated below as NKG2D CAR, NKG2D CAR shMICA/B, NKG2D armored CAR and NKG2D armored CAR shMICA/B, respectively.

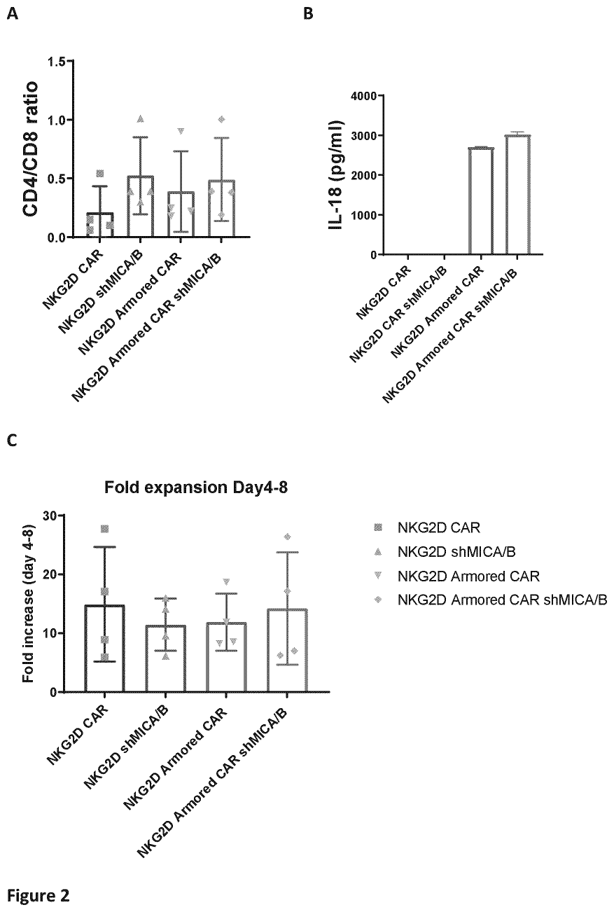

Each construct was used to retrovirally transduce T cells, tag-positive cells were subsequently expanded and purified. When comparing the cultured cells phenotype, it can be observed that the CD4/CD8 ratio of these cells is similar, with the NKG2D CAR showing a slight shift towards a CD8+ phenotype when compared with the other arms (FIG. 2A). The two armored CARs produce active IL-18, whereas the constructs not expressing IL-18 do not (FIG. 2B). Fold expansion in all arms was equivalent (FIG. 2C).

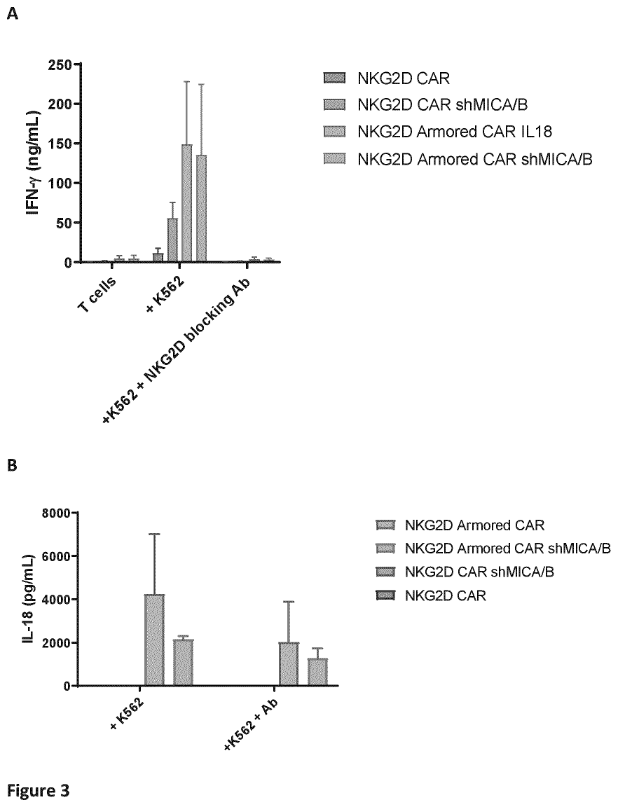

CAR efficacy was tested by coculturing them with K562 cells (a leukemia cell line). Results are shown in FIG. 3. T cells alone secreted almost no interferon-gamma (a measure for activation). When cocultured with K562 cells, all of the CAR T cells secreted IFN gamma. However, the NKG2D CAR with shMICA/B construct secreted more IFN-γ than the NKG2D CAR as such, and both armored CARs secreted even higher levels of IFN-γ. Adding a blocking antibody against NKG2D abolished the IFN-γ secretion of all constructs, proving that the activation is specific and NKG2D-mediated (FIG. 3A). As shown in FIG. 3B, both armored CAR constructs secrete IL-18, while the CAR constructs not expressing IL-18 do not. Unlike IFN-γ the secretion of IL-18 was not completely inhibited by the addition of the NKG2D blocking antibody, although a slight reduction in IL-18 secretion was observed. This indicates that there may be a positive feedback loop to further secrete IL-18 when the NKG2D CAR is activated, although it does not rely on this for the secretion of active IL-18.

### Example 2. Retention of Cytotoxicity in a Repeated Antigen Stimulation Assay

Exhausted T cells are constantly stimulated by chronic inflammatory pathogens or tumor antigens and gradually lose their abilities of antigen recognition, proliferation, and activation, which finally leads to the stepwise loss of effector functions and impaired elimination of viral or tumor antigens. Likewise, it is a known problem of many CAR T cells that they typically become exhausted upon repeated antigen stimulation. To test whether the additional elements in the NKG2D CAR shMICA/B, NKG2D armored CAR and NKG2D armored CAR shMICA/B also delay exhaustion and improve prolonged activity, a repeated antigen stimulation assay (RASA) was performed. To this end, CAR T cells were incubated with PANC-1 cells, a pancreatic tumor cell line for 48 hours and proliferation/survival of PANC-1 was assessed. After 48 hours, the remaining CAR T cells were counted and restimulated with freshly cultured PANC-1 cells, in a 1:1 target: effector ratio. Finally, the process is repeated for a third stimulation to assess growth over a longer time period.

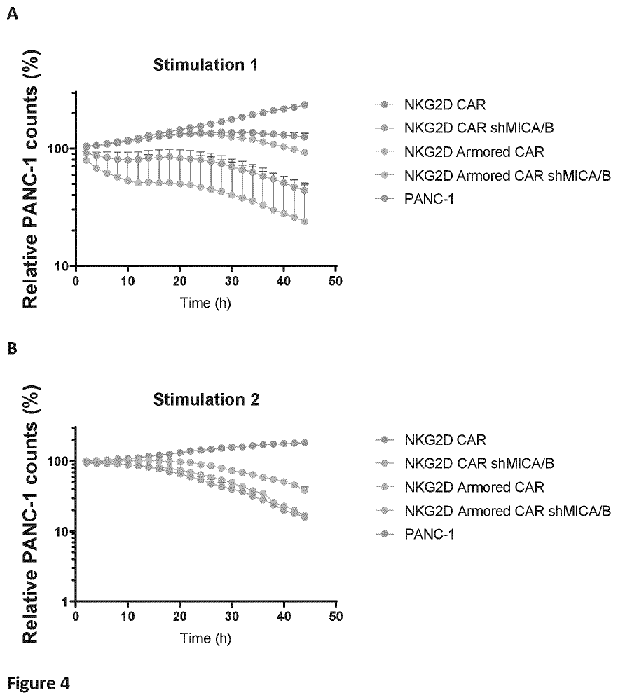

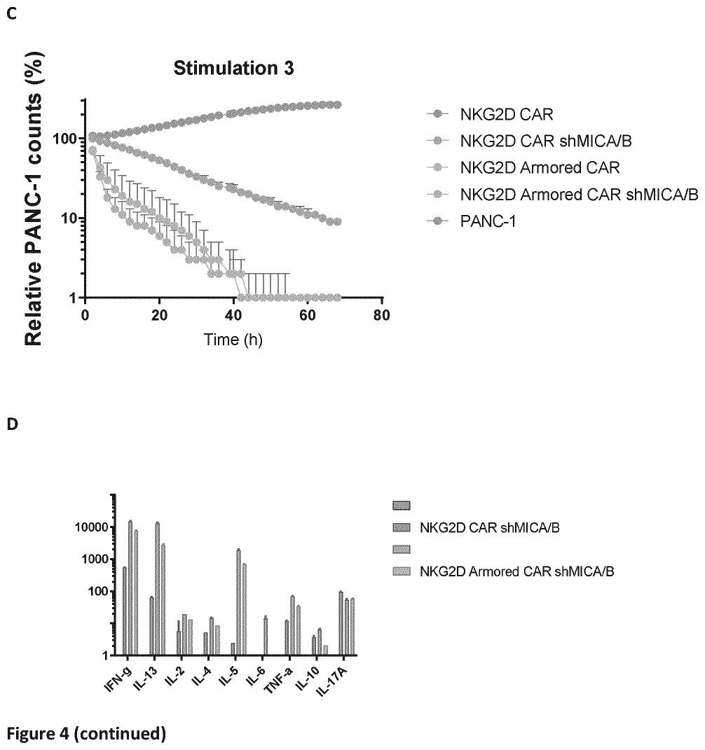

As shown in FIG. 4A, all CARs lyse the cancer PANC-1 cells as compared to PANC-1 cells alone (which continue to proliferate). The NKG2D armored CAR shMICA/B has the highest lytic activity after the first stimulation, with the NKG2D CAR shMICA/B closely behind.

In the second stimulation (FIG. 4B), the NKG2D CAR did proliferate sufficiently to allow for further stimulations and was thus omitted. The remaining 3 constructs all had a significant effect on PANC-1 growth, killing over 50% of the initial PANC-1 cells in 48 hours, whereas PANC-1 cells without a CAR increased their counts. Here, all the NKG2D based CARs showed similar kinetics.

However, the most remarkable result was obtained after the third stimulation (FIG. 4C): both NKG2D armored CARs completely eliminated the PANC-1 cells after 48 hours. This is linked to an increased fold expansion compared to the NKG2D CAR shMICA/B (data not shown). The NKG2D CAR shMICA/B still performed admirably, with over 60% of PANC-1 cells eliminated after 48 hours and over 80% cells eliminated after 72 hours. These results suggest that the NKG2D armored CARs retain their cytotoxicity versus tumor cells upon repeated antigen stimulation, and could have a superior efficacy in vivo following tumor rechallenge.

Next to the armored CAR efficacy in lysis of the PANC-1 cells target cells, cytokine secretion after the third stimulation was markedly different. IL-18 directly influences IFNγ which is indeed increased in all IL-18 secreting cells (FIG. 4D). However, next to IFNγ, a marked increase in both IL-13 and IL-5 was observed indicating a Th1/Th2 profile that drives antibody formation and B cell activation.

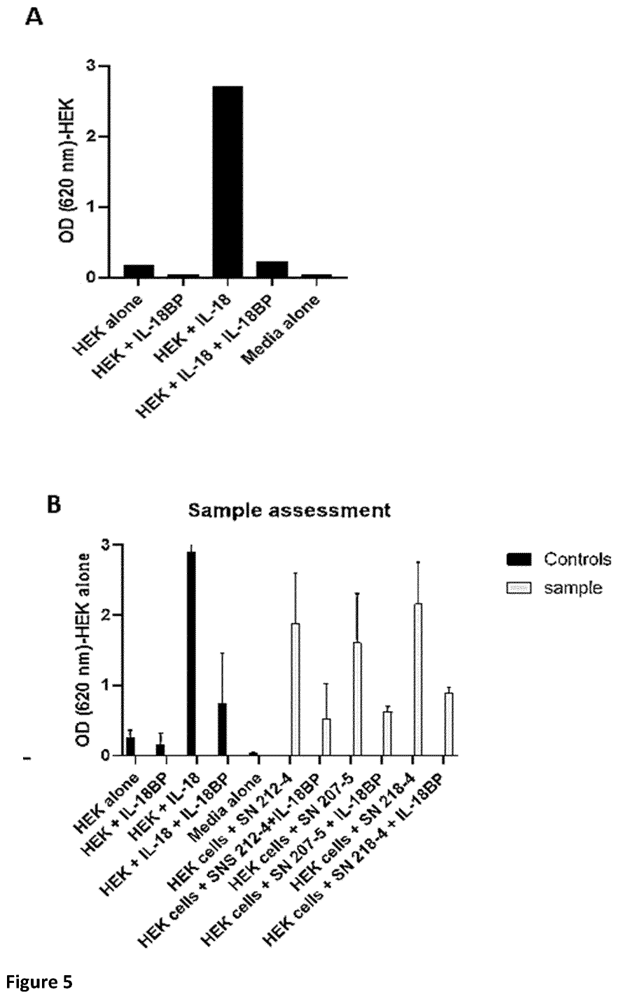

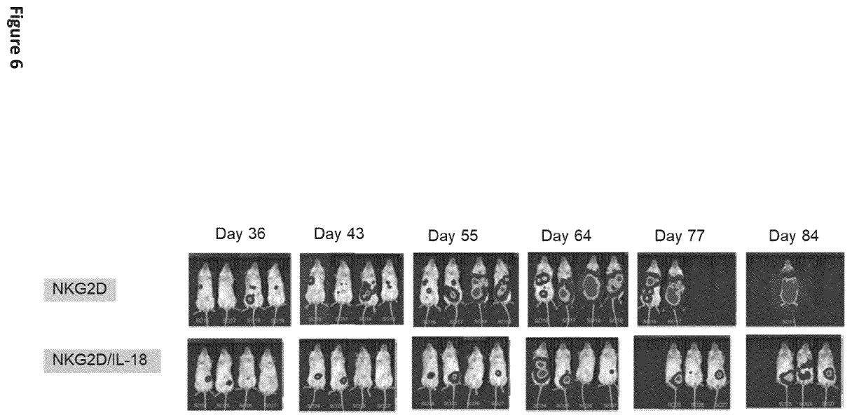

As a control to check that the effect is indeed due to IL-18, functionality of the secreted IL-18 was assessed by developing an assay utilizing a HEK-IL18 cell line. The cells express a secreted embryonic alkaline phosphatase (SEAP) reporter gene under the control of the IFN-β minimal promoter which is fused to five NF-κB and five AP-1 binding sites. Binding of IL-18 to the heterodimeric IL-18 receptor on the surface of these cells triggers a signaling cascade leading to the activation NF-κB and the subsequent production of SEAP. Results are shown in FIG. 5. All armored CAR T cells induced a specific enzymatic response that was inhibited by IL18BP thereby indicating the secreted IL-18 is indeed functional and can interact with its receptor.

### Example 3. In Vivo Performance of NKG2D Armored CARS

To evaluate whether the findings of the RASA are translatable in vivo, NKG2D armored CARs were evaluated in different tumor settings. In the first, NSG mice were orthotopically injected with 200 000 HTC-116 cells (a colon cancer cell line), followed by repeated injections of either NKG2D CAR or NKG2D armored CAR at day 7, 14 and 21. Bioluminescence results are shown in FIG. 6. Control mice all had to be euthanized before day 36, showing that both NKG2D CAR and NKG2D armored CAR provide protection against tumor growth. However, it can be appreciated that tumors develop slower in the group of mice that received the NKG2D CAR also expressing IL-18. This is evident from day 43 onwards, and becomes more pronounced as time progresses. Indeed, the mice that received NKG2D armored CAR also have a survival benefit, as 3 out of 4 reach day 84, with smaller tumors than the 1 out of 4 surviving mouse that received the NKG2D CAR.

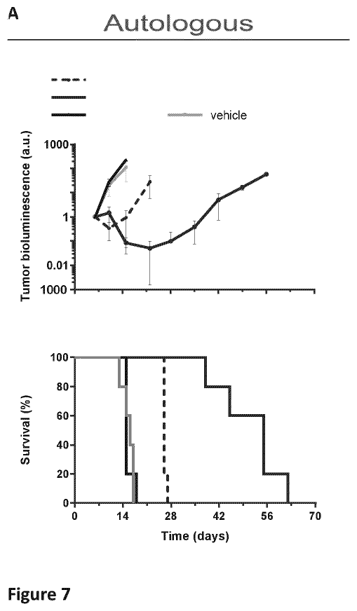

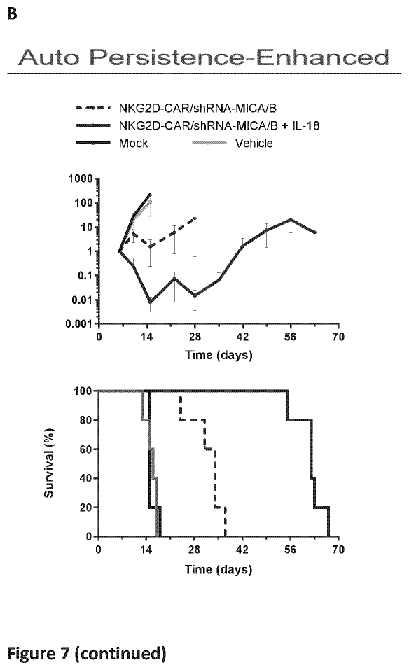

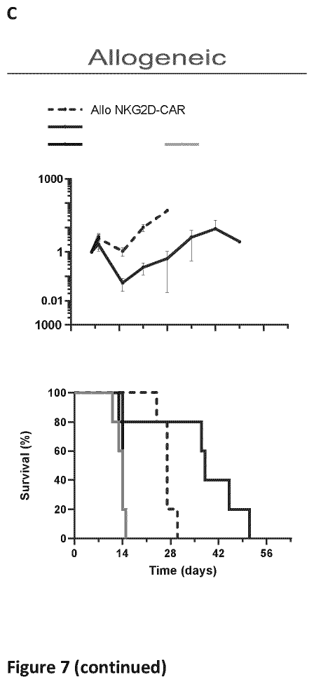

In a second model, the impact of IL-18 incorporation was evaluated in a preclinical model of AML for various CAR-T designs (FIG. 7). Here, the NKG2D CAR was compared against the NKG2D armored CAR to evaluate the addition of IL-18 in an autologous setting (FIG. 7A). Further, the NKG2D CAR shMICA/B was compared against the NKG2D armored CAR shMICA/B to evaluate the contribution of IL-18 in a setting of an autologous CAR with improved persistence (FIG. 7B). Finally, an allogeneic NKG2D CAR (i.e. a CAR that co-expresses a shRNA against CD247, which has reduced to almost absent TCR signalling) was compared to an allogeneic NKG2D armored CAR (FIG. 7C). In all of these settings, the NKG2D CAR clearly has clinical benefit, as it has slowed tumor growth and led to longer survival than the mock or vehicle transduced cells. However, the addition of IL-18 has a huge impact both on tumor growth and survival in all of these settings. Survival is about doubled in both the autologous and the allogeneic setting. In the persistence-enhanced setting (FIG. 7B), survival of the NKG2D CAR shMICA/B is already better (compared with NKG2D CAR in FIG. 7A), but still, the addition of IL-18 has a huge survival benefit. Of note, in all of the groups receiving the armored CAR, all of the mice outlive all the mice having received the non-armored equivalent CAR.

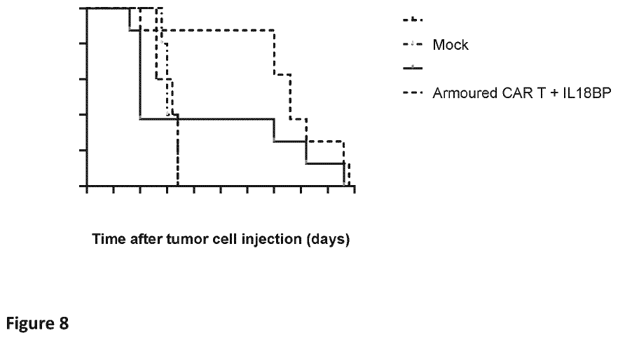

In a third model, NSG mice were injected with THP-1 cancer cells and seven days later infused with vehicle, mock or allogeneic armoured NKG2D CAR T cells. Mice infused with a high dose of allogeneic armoured NKG2D CAR T cells exhibited signs of cytokine release syndrome driven by the secreted IL-18. This effect could be negated by the infusion of IL18BP. Results are shown in FIG. 8.

In conclusion, addition of IL-18 always has beneficial effect on NKG2D CAR function. The effect appears independent of the presence of further elements such as shRNAs, although select shRNAs independently lead to an additional benefit.

1. An engineered cell comprising:
A first exogenous nucleic acid molecule encoding a chimeric NKG2D receptor
a second nucleic acid molecule encoding IL-18.

2. The engineered cell of claim 1, which is an engineered immune cell.

3. The engineered cell of claim 1 or 2, wherein the engineered cell is selected from a T cell, a NK cell, a NKT cell, a stem cell, a progenitor cell, and an iPSC cell.

4. The engineered cell of any one of claims 1 to 3, wherein the first and second nucleic acid molecule are present in one vector.

5. The engineered cell of any one of claims 1 to 4, wherein the IL-18 is secreted.

6. The engineered cell of any one of claims 1 to 5, which has been further engineered to reduce or inactivate TCR signalling.

7. The engineered cell of claim 6, wherein TCR signalling has been inactivated through gene editing.

8. The engineered cell of claim 6, wherein TCR signalling has been reduced through RNA interference.

9. The engineered cell of claim 8, wherein the RNA interference to reduce TCR signalling comprises a RNA interference molecule directed against a TCR receptor complex subunit.

10. A vector comprising:
A first exogenous nucleic acid molecule encoding a chimeric NKG2D receptor
a second nucleic acid molecule encoding IL-18.

11. The engineered cell of any one of claims 1 to 9 for use as a medicament.

12. The engineered cell of any one of claims 1 to 9 for use in the treatment of cancer.

13. A method of treating cancer, comprising administering to a subject in need thereof a suitable dose of cells according to any one of claims 1 to 9, thereby improving at least one symptom.
